(19) United States
(12) Patent Application Publication
(10) Pub. No.: US 2009/0229179 A1
(43) Pub. Date: Sep. 17, 2009

(54) Method of Land Management Involving Microbial Bioassay

(75) Inventors: Kalideen Hafeel, Crawley (AU); Timothy Gerard Quinlan, Margaret River (AU)

Correspondence Address: LAHIVE & COCKFIELD, LLP;FLOOR 30, SUITE 3000, ONE POST OFFICE SQUARE, BOSTON, MA, 02109, US

(73) Assignee: The ERA Farming Company, West Leederville (AU)

(21) Appl. No.: 12/252,964

(22) Filed: Oct. 16, 2008

Related U.S. Application Data
(63) Continuation of application No. 11/886,362.

(30) Foreign Application Priority Data
Mar. 16, 2005 (AU) 2005901277
Sep. 16, 2005 (AU) 2005905142

(51) Int. Cl. A01G 1/00; C12Q 1/06; A01G 17/00

(52) U.S. Cl. 47/58.1 SC; 435/39; 47/58.1 FV

(57) ABSTRACT

A method for land management comprising measuring, as a measure of effectiveness of the method, soil quality of a soil sample collected from land under management by determining fungal; bacterial ratio of said soil sample by substrate induced respiration combined with selective inhibition by multiple soluble inhibitors selected with reference to the land under management to measure soil quality of the land under such management. A nutrient application programme for a specified crop is controlled on the basis of determined fungal bacterial ratio. Such measure can be employed to identify the availability of nutrients such as nitrogen, phosphorus and sulphur to plants as such availability is mediated by soil microorganisms. Action to increase plant availability of nutrients and soil biological activity may be initiated if required. The measurements may also be an indicator of sustainability of land management method. Application of the method to olive and wheat growing is demonstrated.

The invention relates to a method of land management involving the assessment of soil quality as a control parameter in practice of such method.

The intensity of many contemporary land-based primary production systems (agriculture, horticulture, silviculture etc) has led to significant ecosystem degradation in the form of loss of soil biodiversity, nutrient depletion, soil erosion, secondary salinity and adverse effects on water quality. This degradation of soils and associated decreases in productivity and food quality, mean that considerable research attention is being given to assessment of alternative management of primary production systems. Strategies for alternative management range from a more careful accounting of nutrient budgets while still pursuing conventional (fertilizer and pesticide based) management systems, to ‘organic’ management.

Numerous concerns have been raised about the likelihood that strict forms of organic management (referred to simply as ‘organic’ in this application) are unsustainable, since outputs of nutrients may exceed inputs, with subsequent ‘mining’ of soil nutrients. These concerns have led segments of the industry to adopt an approach to sustainable land management, which integrates a focus on organic soil amendments and zero or minimal synthetic pesticide use, with nutrient inputs allowed from inorganic or industrially-produced nutrient sources. In this application, an approach based on this premise is referred to as ‘integrated land management’, biological management or biological farming. Several recent reviews have established that, in many cases, integrated land management or biological farming improves soil quality, particularly by increasing soil organic matter and microbial contents, increasing beneficial microbial activity in soils, and improves soil physical properties. Additional wider benefits may include reducing pesticide residues in food crops, lower export of pollutants (particularly nutrients and greenhouse gases) from managed ecosystems, and lower economic inputs. In addition, both integrated and fully organic management incur lower production costs associated with lower inputs of fossil fuels, manufactured fertilizers and agrochemicals.

An aim of integrated land management is enhancement of biological nutrient cycling to reduce reliance on synthetic nutrient additions and to restore beneficial ecological function to soils degraded by conventional management. With this in mind, considerable effort has been directed by the integrated land management industry towards development and application of alternative soil amendments, or ‘biostimulants’ designed to facilitate or even stimulate soil biological activity. Traditionally, such soil amendments have included: livestock manures; green (crop residue) manures; unprocessed mineral amendments such as lime, rock phosphate or gypsum; or composts prepared from one or more organic source(s). Manuring and compost application promote soil microbial activity directly, primarily by supplying a metabolisable carbon source together with nutrients.

A further distinctive feature of integrated land management practices relates to the type of soil quality data collected and the paradigms for interpretation of such data. In attempts to reflect the underlying values of attaining ecological balance for land under integrated land management, suites of soil analyses such as the Albrecht or Reams systems differ from conventional soil testing by including parameters specifically intended to assess holistic ecosystem health. However, soil chemical analyses may not provide a useful indication of soil quality for land which is managed using integrated or organic paradigms.

It is therefore an object of the present invention to provide a method for land management that allows the assessment of soil quality in a manner which provides a reliable and useful indication of soil quality, particularly from the biological perspective.

With this object in view, the present invention provides—in one aspect—a method for land management comprising measuring, as a measure of effectiveness of the method, soil quality of a soil sample collected from land under management by a microbial bioassay including at least determining fungal:bacterial ratio of said soil sample by substrate induced respiration combined with selective inhibition by multiple soluble inhibitors, wherein said multiple soluble inhibitors are selected with reference to the land under management; and a nutrient application programme for a specified crop growable on the land is controlled on the basis of the determined fungal:bacterial ratio. Such measurement may be employed to identify the availability of nutrients such as nitrogen, phosphorus and sulphur to plants as such availability is mediated by soil micro-organisms. Action to increase plant availability of nutrients may be initiated, if required. The fungal:bacterial ratio soil quality measurement may also be an indicator of sustainability of the land management method.

The soil quality measurement step or aspect of the method reproducibly generates microbial biomass data and fungal:bacterial ratio data having an inhibitor additivity ratio (IAR) of 1.00 (±0.15) where:

IAR=(A−B)+(A−C)/(A−D)

and A is total soil biomass, (A−B) is the difference between total soil biomass and biomass in the presence of fungal inhibitor(s), (A−C) is difference between total soil biomass and biomass in the presence of bacterial inhibitor(s) and (A−D) is the difference between the total soil biomass and biomass in the presence of both fungal and bacterial inhibitors at similar concentrations to A and B. The inhibitors employed are selected or calibrated with reference to soil type and indigenous microflora of that soil type; and/or soil texture in order to avoid inhibition of non-target bacteria or fungi, problematic in prior art methods. Therefore, prior calibration to assess a suitable pair may be required. Calibration involves selecting the best inhibitor pair that shows maximum respiration inhibition on a wide range of soil types; and determining the best inhibitor concentrations that yield the target IAR of 1.00 (±0.15). Such IAR indicates that the inhibitors have acted only upon the target organisms such that measured fungal:bacterial ratio has acceptable reliability. An IAR of greater than 1.00 (±0.15) signifies non-target, and less than 1.00 (±0.15) inadequate inhibition.

Fungal inhibitors, such as cycloheximide, and bacterial inhibitors, such as kanamycin, as selected by the Applicants for tests on a Western Australian soil at the University of Western Australia, are applied to soil samples in multiple different concentrations. Concentrations may be adapted to different suites of commonly occurring soil microorganisms such as heterotrophic bacteria and fungi, such as arbuscular mycorrhizal fungi, fluorescent pseudomonads and actinomycetes and according to the suites of microbes actually inhabiting the land under management. Microbe nature varies with soil type and soil characteristics. A total of 7 soil treatments to establish inhibitor efficiency has been found beneficial. The inhibitors are applied to the soil sample, in accordance with the method, in solution form. Talc or similar substrates are advantageously avoided as it is not necessary when inhibitors are applied in solution form.

Glucose or similar carbon source or respiratory substrate for microbes is added to each soil sample during the analysis. The carbon source may be added following addition of inhibitor.

Moisture in the sample is controlled to ensure proper dispersion of inhibitor and glucose and aerobic conditions.

The measurement of fungal:bacterial ratio may support or supplement measures of total microbial biomass as a step in assessing soil biological activity as part of the land management method.

Soil quality data obtained in accordance with the land management method, which may—for example—be conducted in accordance with Australian Patent Application No. 57966/01, the contents of which are hereby incorporated by reference, form a further aspect of the invention which may be compared with objective standards for the soil in the managed land and a nutrient application programme maintained or varied to reduce any adverse variations from these objective standards. Reliable soil quality data assists this. Such nutrient application programme is preferably a biologically acceptable nutrient application programme.

In such embodiment, the present invention provides a biological method of managing crop production comprising the following steps:

a) selecting an area to be farmed with a specified crop;

b) evaluating said area, which evaluation includes conducting physical and/or chemical analysis of the soil environment of said area;

c) determining objective standards for soil and crops in said area and a biological nutrient application programme for the area aimed at achieving those of the objective standards which are specific to the specified crop, the programme consisting of application of biologically acceptable components not known to cause genetic modification of plants;

d) conducting the biological nutrient application programme;

e) conducting testing of soil and specified crops in the area for measuring compliance with said objective standards; and

f) adjusting the biological nutrient application programme, as necessary, to reduce adverse variations from said objective standards and wherein the biological nutrient application programme is conducted to stimulate microbial activity to promote plant availability of nitrogen and in response to microbial bioassay data, most advantageously fungal:bacterial ratio data for soil samples from the area to be farmed with the specified crop. This ratio is determined by a method, as described above, in which the multiple soluble inhibitors are selected with reference to the area to be farmed with the specified crop.

Nutrients should, more desirably must, be introduced to the soil only in biologically acceptable form, the objective being to implement a nutrient application programme, controlled to increase the biological activity of soil in the land while maintaining ecological sustainability. Organic form may be acceptable in certain circumstances. Thus, phosphorus may be introduced in the form of phosphate rock and nitrogen may be introduced through use of animal waste products and fish based products. Nitrogen control, in terms of controlling nitrogen input to land under management, is important. While nitrogen is essential to crop growth, excess nitrogen can create problems by increased susceptibility of crop to disease and insect attack. Nutritional content may also be reduced. These issues may be addressed by growing of plants (such as clover and legumes) and enhancement of nitrogen fixing microbes, enhancing nitrogen fixation from the atmosphere. Nutrients may also, and advantageously, be supplied by biological fertilizer products such as that available under the trade mark ERAPHOS, subject of Australian Patent Application No. 18362/01, the contents of which are hereby incorporated by reference.

The land management method, as described in Australian Patent Application No. 57966/01 is an integrated land management method concerned with ecological sustainability which may be applied to the growing of various crops such as wheat and olives being examples of specified crops. The method may also be used to assess potential for various crops to be grown in the land under management. For example, data generated by the method may assist in identifying crops suitable for growing in the land under management.

Such cash crops may be grown in an initially nitrogen deficient soil, thus warranting a strategy to improve biological activity and better plant availability of nitrogen through biological mineralization of nitrogen. Soil bioassay to assess total microbial biomass and the fungal:bacterial ratio is included, advantageously as above described, to assess the efficacy of the method to improve biological activity and better plant availability of nitrogen and organic matter through sufficient humification of the soil. Using such data, a biological nutrient application programme may be controlled to achieve these objectives. This forms a further aspect of the present invention. In the case of olives, a preferred range of fungal:bacterial ratio is 2:1 to 5:1 so, if a soil is fungally deficient, the method will include steps, such as administration of fungal inocula and fungal enriched compost to increase the ratio towards the preferred range. Conversely, if a bacterial deficiency is identified a strategy to augment soil biological activity may be implemented. Microbial inocula and bacteria rich fertilizers such as compost tea or fish emulsion, may be applied to increase microbial activity.

An additional step of mycorrhizal bioassay, such as by measuring mycorrhizal inoculum potential, may be included within the method or as an additional measurement of soil quality. In particular, the presence of mycorrhizal fungi is an indicator of the balance of soils and the sustainability of the land management method. Changes in mycorrhizal inoculum potential may also be tracked, with fungal:bacterial ratio, in response to changes in land management techniques. The nutrient application programme is then controlled as a function of fungal:bacterial ratio and mycorrhizal inoculum potential.

Mycorrhizal inoculum potential may be measured in the field, rather than through the agency of laboratory testing. The plant roots are cleared and stained as described, for example, Koske, R E, Gemma, J N (1989) A Modified Procedure for Staining Roots to detect V-A Mycorrhizas, Mycological Research 92: 486-488, Phillips, J M, Hayman, D J (1970) Improved Procedures for Clearing Roots and Staining Parasitic and Vesicular Arbuscular Mycorrhizal Fungi for Rapid Assessment of Infection, Transactions of the British Mycological Society 55: 159-161, the contents of which are hereby incorporated by reference. Stained roots are laid down on microscopic slides or gridded Petri dishes and mycorrhizal colonization (inoculum potential) scored under a microscope.

Simple equipment, avoiding expensive laboratory equipment such as autoclaves—for example by using electric pressure cookers, allows examination of the mycorrhizal status of any crop species in its native environment and in its active growth phase. This information can be used for growth model construction and simulation. By confining the tests to the field, time may be saved in sample handling and processing. The direct testing of crop roots is advantageous over collecting paddock soils and performing mycorrhizal bioassays in glasshouse-grown plants, as the plant and the environmental conditions are totally different in glasshouse bioassays.

The practice of the method of the invention may be more fully understood from the following examples of application of methods of the invention. The description is illustrative of the methods rather than limiting and its application to wheat farming and olive growing situations is demonstrative of only two types of crop to which the method could be directed. Other crops, such as palm fruit to be used in the production of palm oil, may also be managed in accordance with the method of the invention.

### EXAMPLES

### Example 1

Soil samples were obtained from a wheat paddock. Measurement of soil quality was conducted in accordance with the following protocol in accordance with the method of the invention.

1. Using 30 ml McCartney bottles, prepare the following experimental trials in duplicates or triplicates:

There are seven soil sample treatments, this number being beneficial in the practice of the method.

TABLE 1
Soil sample treatments.
Column 1 shows 2 g portions of the soil sample in 8 separate tubes.
Column 2 shows the treatment each tube receives. The moisture content
of the sample in each tube at this stage is about 15% water
holding capacity.

| Tube | |
|---|---|
| name | Treatment |
| A | None |
| B | Cycloheximide (fungal inhibitor); Rate, 2 mg inhibitor/tube |
| C | Kanamycin (bacterial inhibitor); Rate, 3 mg inhibitor/tube |
| D | Cycloheximide and Kanamycin mixed together at rates |
| | shown in columns B & C |
| E | Cycloheximide; Rate, 3 mg inhibitor/tube |
| F | Kanamycin; Rate, 2 mg inhibitor/tube |
| G | Cycloheximide and Kanamycin mixed together at rates |
| | shown in columns E & F |
| H | Cycloheximide and Kanamycin mixed together at rates |
| | shown in columns B & F |

The inhibitors are mixed with water to form solutions of inhibitors, absent talc or other substrate, and applied at rates of 200 μL/tube.

2. Incubate for 1 hr at room temperature

3. Add glucose at rates of 50 μL/tube (8% solution, w/v) and seal the bottles with rubber septa.

5. Incubate at 25° C. for 4 hrs in the dark

6. Calibrate the IR gas analyzer using 0.1 ml, 0.2 ml and 0.3 ml 4.95% ultra pure standard CO₂ gas

7. Syringe out 1 ml portions of the headspace gas from the sample bottles and inject into the calibrated gas analyser

8. Measure the peak heights of standards and samples

9. Calculate the biomass for each sample using the following equation of Anderson, J and Domsch, K “Measurement of Bacterial and Fungal Contributions to Respiration of Selected Agricultural and Forrest Soils”, Can J Microbiol, Vol 21, 1975, p 314.

μg CO₂—Cg⁻¹soil=((Aₛₐₘₚₗₑ×10000/B_{std})/10³×V×K))−((A_{blank}×10000/B_{std})/10³×V×K))

where:

Aₛₐₘₚₗₑ=Peak height (mm) of sample

A_{blank}=Peak height (mm) of blank bottle

B_{std}=Peak height (mm) of standard gas (1% CO₂)

V=Head space volume of bottle

K=Conversion constant for μl to μg of CO₂—C (assuming 1 atmosphere pressure: 1.7995 μg CO₂=1 μl CO2 at 25° C. and 1 atmosphere=0.4908 μg CO₂—C)

10. Calculate the parameters, and pick up the most acceptable fungal bacterial ratio, i.e. the one with an IAR closest to 1.00, from one of the following 3 datasets:

TABLE 2
Datasets generated from the incubation trials.

| | | | | Fungal | Inhibitor |
|---|---|---|---|---|---|
| | | Fungal | Bacterial | bacterial | additivity ratio |
| Dataset | Treatment* | biomass | biomass | ratio | (IAR) |
| 1 | A B C D | (A − B) | (A − C) | (A − B)/ | (A − B) + |
| | | | | (A − C) | (A − C)/ |
| | | | | | (A − D) |
| 2 | A E F G | (A − E) | (A − F) | (A − E)/ | (A − E) + |
| | | | | (A − F) | (A − F)/ |
| | | | | | (A − G) |
| 3 | A B F H | (A − B) | (A − F) | (A − B)/ | (A − B) + |
| | | | | (A − F) | (A − F)/ |
| | | | | | (A − H) |

*Letters A through to H correspond to tube names in Table 1.

TABLE 3
Example of an actual dataset from a paddock soil sample.

| | Total | | | Fungal | |
|---|---|---|---|---|---|
| | microbial | Fungal | Bacterial | bacterial | |
| Dataset | biomass | biomass | biomass | ratio | IAR |
| 266A | 25.6 | 8.9 | 3.5 | 25 | 1.11 |
| 266B | 25.6 | 10.1 | 4.7 | 2.1 | 1.19 |
| 266C | 25.6 | 8.9 | 4.7 | 1.9 | 1.28 |

Biomass values are in μg/g dry soil

It is clear from Table 3 that dataset A has yielded an IAR 1.11, within the acceptable range, and hence the biomass and fungal bacterial ratio of this dataset are the most acceptable numbers and may be used in the control of an integrated land management method.

### Example 2

Olive trees were planted in order to establish an olive grove. The grove lacked legume species, thus reducing photosynthetic carbon root exudates that may act as food source for different species of microorganisms. Accordingly, action was proposed to enhance growth of diverse micro-organisms in order to optimise plant growth. Specifically, optimal fungal:bacterial ratios for olive growth lie in the range 2:1 to 5:1.

Fungal:bacterial ratio measurements made by a method as above described, confirmed plant tissue testing showing low levels of nitrogen, as an indicator of insufficient microbial activity to mineralise enough nitrogen to sustain crop growth. In addition, the organic matter in the soil of the grove was insufficiently humified. One soil sample had a fungal:bacterial ratio of 1.2:1 and other soil samples had lower fungal content and indicating a potential fungal deficiency. The range for microbial biomass across the samples was 26.1-45.1 μg/g and that for mycorrhizal inoculum potential was 29 to 46%.

In order to address these issues, fertilisation strategy was altered. A fungal dominant or fungus inoculated compost blend was applied, as a fungal augmentation strategy for adding sufficient quantity of organic matter to the region of soil around each olive tree to sustain growth for a considerable period. Mulching with woodchips supplemented the organic matter. Such mulch is primarily composed of cellulose and lignin. Organisms well adapted to decompose such material are saprophytic fungal species with which the compost was enriched. On decomposition, the product simple sugars and carbohydrates act as food source for actinomycetes and bacterial species. Predation from grazing organisms such as nematodes and protozoa (which mainly consume bacteria) will cause release of—among other compounds—organic nitrogen to be taken up by the olive roots. Accordingly, the total biomass of fungi and bacteria in the soil is the main source of plant available nitrogen.

Accordingly, promotion of such nitrogen cycling and mineralisation mechanisms was the objective for management of the olive grove. Integration of microbial inoculums such as compost teas, fungal, bacterial and protozoa infusions was therefore employed in the nutritional program to boost biological activity and plant availability of nitrogen. Compost tea and fish hydrolysate were applied in combination. Compost tea introduced a diverse range of beneficial bacterial fungi and protozoa species in high population.

Specifically, an annual nutrient applicant programme involved the following stages:

Stage 1

Fertigation initiated of blended molasses, soluble kelp powder (sold under Seagold trade mark) and fish hydrolysate. Such products could be applied mixed, separately or sequentially. The administration rates of these products were:

| | |
|---|---|
| Molasses | 3-5 L/Ha |
| Kelp Powder | 300 g/Ha |
| Fish Hydrolysate | 5-10 L/Ha |

Stage 2

A first application of fungal dominant compost tea occurred within 3-5 days of applying the molasses/kelp powder/fish hydrolysate blend in Stage 1. Such application was as a soil drench to the base of each tree at a rate of 50-100 L/Ha. Addition of a commercial Azotobacter inoculum to the compost tea introduced nitrogen fixing bacteria to the soil system.

Post Compost Tea (CT)

After applying the above CT and subsequent applications, a follow up food source application was made within 3-5 days. This was selected to be the same as the above blend at the same rates to provide continual food sources for the organisms.

Protozoa or Hay Infusions

An organic source of hay was used for this preparation. The root crown and stalk of hay are loaded with protozoa. As the hay dries down the protozoa hibernate until moisture levels rise. A simple preparation was made by infusing a small amount of hay into tepid water and leaving for 5-7 days. Protozoa consume bacteria and produce ammonium as a waste product which is rapidly converted into nitrate (nitrogen mineralization). Such a preparation was applied after the first CT application to enhance protozoa activity.

Remainder of Growing Season

Maintenance fertigations of CT at a rate of 50 L/Ha at monthly intervals alternating with food source applications and protozoa infusions if nitrogen stress is still present. Advantageously, at least three of these maintenance applications occur after the initial inoculation. Foliar or trichoderma sprays may be mixed with CT application. The efficacy of trichoderma as a biocontrol of plant pathogens may be enhanced if used in combination with compost tea.

As growth progresses, determined fungal:bacterial ratios are expected to increase towards the target tungal:bacterial ratio range of 2:1 to 5:1. Plant tissue analysis for nitrogen is expected to confirm increasing nitrogen availability to the olive trees.

Modifications and variations to the method of the present invention may be apparent to the skilled reader of this disclosure. Such modifications and variations are within the scope of the present invention.

1. A method for land management comprising measuring, as a measure of effectiveness of the method, soil quality of a soil sample collected from land under management by a microbial bioassay comprising at least determining fungal:bacterial ratio of said soil sample by substrate induced respiration combined with selective inhibition by multiple soluble inhibitors wherein said multiple soluble inhibitors are selected with reference to the land under management and a nutrient application programme for a specified crop growable on the land is controlled on the basis of determined fungal:bacterial ratio.

2. The method of claim 1 wherein said multiple soluble inhibitors are calibrated with reference to microflora indigenous to the soil of land under management.

3. The method of claim 1 or 2 wherein said nutrient application programme introduces biologically acceptable nutrients to the land under management.

4. The method of any one of the preceding claims wherein nitrogen input to the land under management is determined as a function of measured fungal:bacterial ratio data.

5. The method of any one of the preceding claims wherein the specified crop is selected from wheat, olives and palm fruit.

6. The method of any one of the preceding claims wherein Inhibitor Additivity Ratio (IAR) is reproducibly 1.00 (±0.15).

7. The method of any one of the preceding claims further comprising the step of measuring at least one of total microbial biomass and mycorrhizal inoculum potential as a measure of soil quality.

8. The method of claim 7 wherein said nutrient application programme is controlled as a function of fungal:bacterial ratio and mycorrhizal inoculum potential.

9. The method of any one of the preceding claims wherein the nutrient application programme is controlled to increase biological activity of soil in the land under management.

10. The method of claim 7 wherein mycorrhizal inoculum potential is measured in the field.

11. The method of claim 10 wherein crop roots are used as test material.

12. The method of any one of the preceding claims wherein, on determination of a fungal deficiency, fungal inocula and mulching form a fungal augmentation strategy for addressing the deficiency.

13. The method of claim 12 wherein said fungal augmentation programme involves saprophytic fungal species.

14. A method of growing olives in an olive grove comprising measuring soil quality of a soil sample collected from said olive grove by determining fungal:bacterial ratio of said soil sample by substrate induced respiration combined with selective inhibition by multiple soluble inhibitors wherein said multiple soluble inhibitors are selected with reference to olive grove soil microflora and a nutrient application programme for olive trees in the grove is controlled on the basis of determined fungal:bacterial ratio.

15. A method of growing wheat comprising measuring soil quality of a soil sample collected from a wheat paddock by determining fungal:bacterial ratio of said soil sample by substrate induced respiration combined with selective inhibition by multiple soluble inhibitors wherein said multiple soluble inhibitors are selected with reference to wheat paddock soil microflora and a nutrient application programme for wheat in the paddock is controlled on the basis of determined fungal:bacterial ratio.

16. A method for measuring soil quality comprising measuring fungal:bacterial ratio of a soil sample by substrate induced respiration combined with selective inhibition by multiple soluble inhibitors wherein said multiple soluble inhibitors are selected with reference to microflora indigenous to the sampled soil for use in accordance with a method as claimed in any one of the preceding claims.

17. The method of any one of the preceding claims wherein the nutrient application programme is ecologically sustainable.

18. A biological method of managing crop production comprising the following steps:
a) selecting an area to be farmed with a specified crop;
b) evaluating said area, which evaluation includes conducting physical and/or chemical analysis of the soil environment of said area;
c) determining objective standards for soil and crops in said area and a biological nutrient application programme for the area aimed at achieving those of the objective standards which are specific to the specified crop, the programme consisting of application of biologically acceptable components not known to cause genetic modification of plants;
d) conducting the biological nutrient application programme;
e) conducting testing of soil and specified crops in the area for measuring compliance with said objective standards; and
f) adjusting the biological nutrient application programme, as necessary, to reduce adverse variations from said objective standards and wherein the biological nutrient application programme is conducted to stimulate microbial activity to promote plant availability of nitrogen and in response to microbial bioassay data.

19. The method of claim 18 wherein said microbial bioassay comprises at least determining fungal:bacterial ratio of a soil sample from the area by substrate induced respiration combined with selective inhibition by multiple soluble inhibitors selected with reference to the area to be farmed with said specified crop.
